(19) United States
(12) Patent Application Publication
(10) Pub. No.: US 2018/0235779 A1
(43) Pub. Date: Aug. 23, 2018

(54) TWO-PART PROSTHETIC SOCKET AND METHOD OF MAKING SAME

(71) Applicant: Ralph Wayne Dudding, Imperial, PA (US)

(72) Inventor: Ralph Wayne Dudding, Imperial, PA (US)

(21) Appl. No.: 15/900,662

(22) Filed: Feb. 20, 2018

Related U.S. Application Data
(60) Provisional application No. 62/460,354, filed on Feb. 17, 2017.

(51) Int. Cl. A61F 2/50; A61F 2/80

(52) U.S. Cl. CPC A61F 2/5046; A61F 2002/5049; A61F 2002/5007; A61F 2/80

(57) ABSTRACT

A two-part prosthetic socket which includes an inner socket component having an inner profile substantially complementary to a profile of a residual limb of a patient, and an outer socket component configured to releasably attach about an outer surface of the inner socket component is disclosed. The inner profile of the inner socket component may be determined from digital data output from a medical imaging scan of the residual limb, which provides a three-dimensional digital profile of the residual limb that includes information on the size and location of at least bone and bone spurs, muscle, scar tissue, and neuroma. The digital data may further include a designed operating range for the size and shape of the three-dimensional digital profile of the residual limb based on such information. The two-part prosthetic socket may be manufactured using additive manufacturing, wherein the inner socket may be formed of a flexible material and the outer socket may be formed of a rigid material.

### CROSS-REFERENCE TO RELATED APPLICATIONS

This application claims the benefit of U.S. provisional patent application No. 62/460,354 filed on Feb. 17, 2017.

### BACKGROUND OF THE INVENTION

### Field of the Invention

This invention pertains generally to a socket assembly for a prosthetic device, and more specifically to a two-part socket assembly having an outer socket for connection to the prosthetic device and an inner socket which acts as an orthotic device, wherein computed tomography (CT), MRI, or other advanced imagery techniques that assess the inner characteristics of the residual limb are used to inform the socket design.

### Background of the Invention

Leg prosthesis sockets are generally made from a positive replica of the person's residual limb. These positive molds are made from physical molds, simulations based upon measurements, or from blue-light scanning the surface of the residual limb. The prosthetic socket is then built up by laminating layers of polymer material over the positive mold or constructed based on a computer model derived from the data collected by the CAD/CAM system. The entire process from taking initial measurements of the residual limb to final delivery of the prosthesis to the patient can take weeks or more and is labor intensive.

This approach is based on a fundamentally flawed assumption; the socket is constructed based upon a static model of the residual limb. The residual limb changes in shape and volume throughout the day, from day to day, and from week to week. Similarly, as patients use their residual limbs with their prosthetic devices, the residual limbs may build muscle and/or portions of the residual limb may change shape due to stresses placed on it during use. As a patient ages, the residual limb will continue to change in response to continued use and environmental conditions.

Further, there are superficial changes that greatly influence the usability and comfort of a prosthesis, such as development of sores and blisters as well as retention of heat and moisture at the skin surface. Depending upon the degree of change and discomfort, a new socket may be needed due to the degree of discomfort the patient experiences. On average, a patient with a new prosthetic will require a new fitting often, possibly as often as every couple of months. This gradually slows to requiring a new fitting every year and even later on, every couple of years. Using currently available techniques for making prosthetic sockets, each fitting and manufacture of a new prosthesis socket may require several weeks.

When refitting a patient for a prosthesis, one fundamental mistake often made is basing the new measurements upon the original positive mold of the residual limb under the assumption that changes in shape are uniformly distributed. These assumptions are nearly always incorrect and lead to another ill-fitting socket.

Another undesirable characteristic of current leg prostheses are their weight. Prosthetic weight is a critical issue because an amputee must exert much more effort to walk with a prosthetic due to having less leg muscle available to assist in walking. Weight is one of the major reasons why a prosthetic device is rejected by the patient. The overall prosthesis should be designed with an aim towards using as little weight as possible.

Accordingly, there is a pressing need for the development of new technologies that minimize prosthesis-associated discomfort, allowing patients to return quickly to the highest quality of life and activity levels achievable. In particular, the prosthetic field would benefit greatly from improved socket designs that can accommodate inevitable changes in limb structure and configuration (e.g., muscle, bone, scar tissue sizes and positions) and are highly customizable to an individual patient's needs. This should be coupled with lightweight materials and a rapid method of manufacture that allows for said customization.

The presently disclosed invention provides a two-part prosthetic socket that overcomes many of the shortcomings of the prior art. The two-part prosthetic socket disclosed herein is more easily customized, weighs less, and may be rapidly manufactured and delivered to a patient.

### SUMMARY OF THE INVENTION

In one aspect, the present invention provides a method for providing a prosthetic inner socket.

In one embodiment, the prosthetic inner socket may be prepared by a process that includes the following steps:
a) receiving digital data collected from an imaging modality regarding three-dimensional (3D) properties of a residual limb, wherein the digital data includes internal and external characteristics of the residual limb;
b) processing said digital data to develop a digital profile of an inner socket; and
c) manufacturing the inner socket based on the digital data.

Within the context of this embodiment, the imaging modality may be, for example, X-ray, CT, MRI, or combinations thereof.

The digital data collected from the imaging modality may include internal characteristics and external dimensions. Examples of internal characteristics include, but are not limited to, scar tissue thickness and location, neuroma size and location, bone spur size and location, muscle location, bone location, and fatty tissue location.

Within the context of this embodiment, digital data may be collected from the residual limb residual limb is at rest and when the residual limb is in weight-bearing use.

The data collected from the imaging modality may then be processed to develop an inner digital profile of an inner socket.

In some embodiments, the processing of the data may be done by artificial intelligence that combines structured and unstructured data from the imaging modality, feedback from the patient who will utilized said prosthetic socket, biometric information (for example, blood pressure, body weight, fat composition, and combinations thereof), and data collected from a medical practitioner (for example, gait analysis, medical examination, and biodynamic examination).

The inner digital profile may be based on characteristics of the residual limb identified in the digital data. For example, the lattice structure of the inner socket may be varied to accommodate both internal characteristics and external dimensions of the residual limb. The lattice structure, formed from cells and ligaments, may display a variety of patterns, including, but not limited to, square, circle, triangular, hexagonal, and combinations thereof.

An inner socket will then be manufactured based on the inner digital profile of the inner socket and have a shape complementary of the residual limb of a patient.

Within the context of this embodiment, the inner socket may be formed from a flexible material. For example, in some embodiments, an elastomeric material is used. Examples of suitable materials include, but are not limited to, styrenic block copolymers, polyolefinelastomers, vulcanizates, polyurethanes, copolyester, polyamides silicon, and combinations thereof.

The inner socket may be manufactured by additive manufacturing methods, for example, by 3D printing.

In another aspect the present invention provides an inner socket of a prosthesis, the design of which is informed by digital data collected from an imaging modality.

The imaging modality may be, for example, X-ray, CT, MRI, or combinations thereof. The digital data collected from the imaging modality may include external dimensions and internal characteristics of the limb, for example, scar tissue thickness and location, neuroma size and location, bone spur size and location, muscle location, bone location, fatty tissue location, and combinations thereof. The digital data may be collected from the residual limb when the residual limb is at rest and when the residual limb is in weight-bearing use.

The inner socket may be made of a flexible material, for example, an elastomeric material. Examples of suitable materials include, but are not limited to, styrenic block copolymers, polyolefinelastomers, vulcanizates, polyurethanes, copolyester, polyamides silicon, and combinations thereof. The inner socket may be manufactured by additive manufacturing methods, for example, by 3D printing. The material may be manufactured into a lattice structure wherein the structure at any particular location on the inner socket is complementary to the residual limb and is informed by the digital data collected on the residual limb. The pattern of the lattice structure may be further varied, for example, the pattern may be square, circle, triangular, hexagonal, or any combinations thereof.

### BRIEF DESCRIPTION OF THE DRAWINGS

Aspects, features, benefits, and advantages of the embodiments herein will be apparent with regard to the following description, appended claims, and accompanying drawings. In the following figures, like numerals represent like features in the various views. It is to be noted that features and components in these drawings, illustrating the views of embodiments of the presently disclosed invention, unless stated to be otherwise, are not necessarily drawn to scale.

FIG. 1 is a prosthetic device as currently described and known in the art;

FIG. 2 is a computed tomography scan of a residual limb;

FIG. 3A is a CT scan of a residual limb indicating a common anatomical cause of pain that may be assessed by CT;

FIG. 3B is a CT scan of a residual limb indicating a common anatomical cause of pain that may be assessed by CT;

FIG. 3C is a CT scan of a residual limb indicating a common anatomical cause of pain that may be assessed by CT;

FIG. 3D is a CT scan of a residual limb indicating a common anatomical cause of pain that may be assessed by CT;

FIG. 4A is a CT scan of a residual limb when the residual limb is non-weight bearing;

FIG. 4B is a CT scan of a residual limb when the residual limb is weight bearing;

FIG. 5 is a side view of one embodiment of an inner socket component as disclosed herein;

FIG. 6A is one example of a lattice design that is more compliant;

FIG. 6B is one example of a lattice design that is less compliant; and

FIG. 7 is a diagram depicting a patient wearing the two-part prosthetic socket on a residual limb.

### DETAILED DESCRIPTION OF THE INVENTION

In the following description, the present invention is set forth in the context of various alternative embodiments and implementations involving a two-part prosthetic socket and methods of manufacturing the same. Wide the following description discloses numerous exemplary embodiments, the scope of the present patent application is not limited to the disclosed embodiments, but also encompasses combinations of the disclosed embodiments, as well as modifications to the disclosed embodiments.

Various aspects of the two-part prosthetic socket may be illustrated by describing components that are coupled, attached, and/or joined together. As used herein, the terms “coupled,” “attached,” and/or “joined” are interchangeably used to indicate either a direct connection between two components or, where appropriate, an indirect connection to one another through intervening or intermediate components. In contrast, when a component is referred to as being “directly coupled,” “directly attached,” and/or “directly joined” to another component, there are no intervening elements shown in said examples.

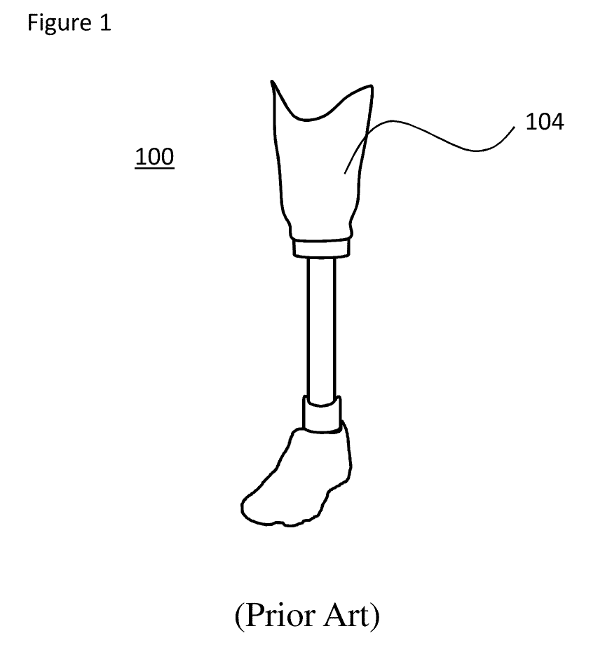

In one aspect, the present invention provides a prosthetic socket for a residual limb of an amputee. Conventional prosthetics often consist of a single socket which is based on a static model of a patient's residual limb. One example of a conventional, prior art prosthetic limb 100 is shown in FIG. 1. Sockets 104 for such prior art limbs are typically fashioned from a physical external mold of the patient's residual limb or from data collected from light-scanning the surface of the residual limb. Over time, changes in anatomy and normal wear and tear cause the socket 104 to become ill-fitting on the residual limb, leading to numerous problem and a lower quality of life. Ultimately, a prosthesis is often rejected due to the discomfort experienced when wearing it.

At the heart of the problem is that the initial socket 104 is constructed based on a static model of the exterior of the residual limb and does not take into account internal anatomy of the residual limb or anatomical changes in the residual limb that occur over time or in different weight-bearing scenarios.

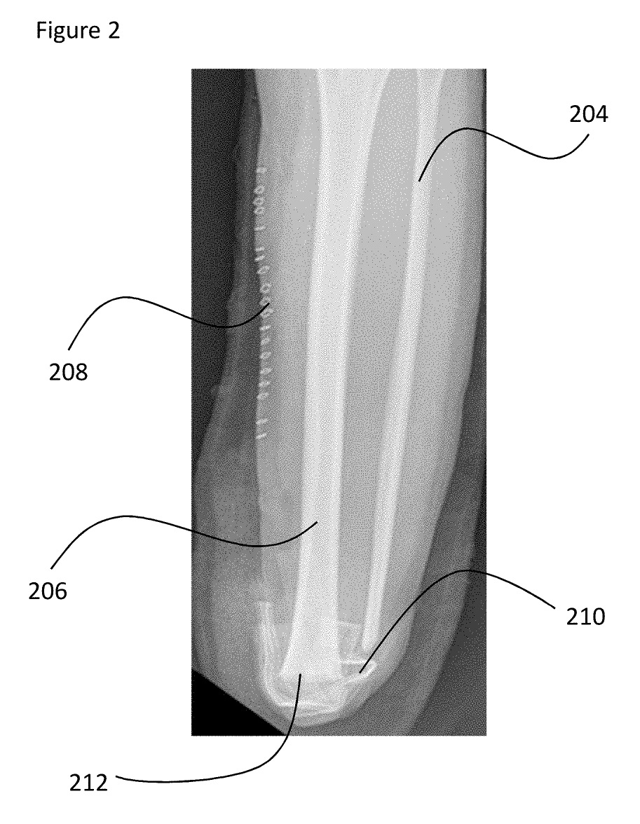

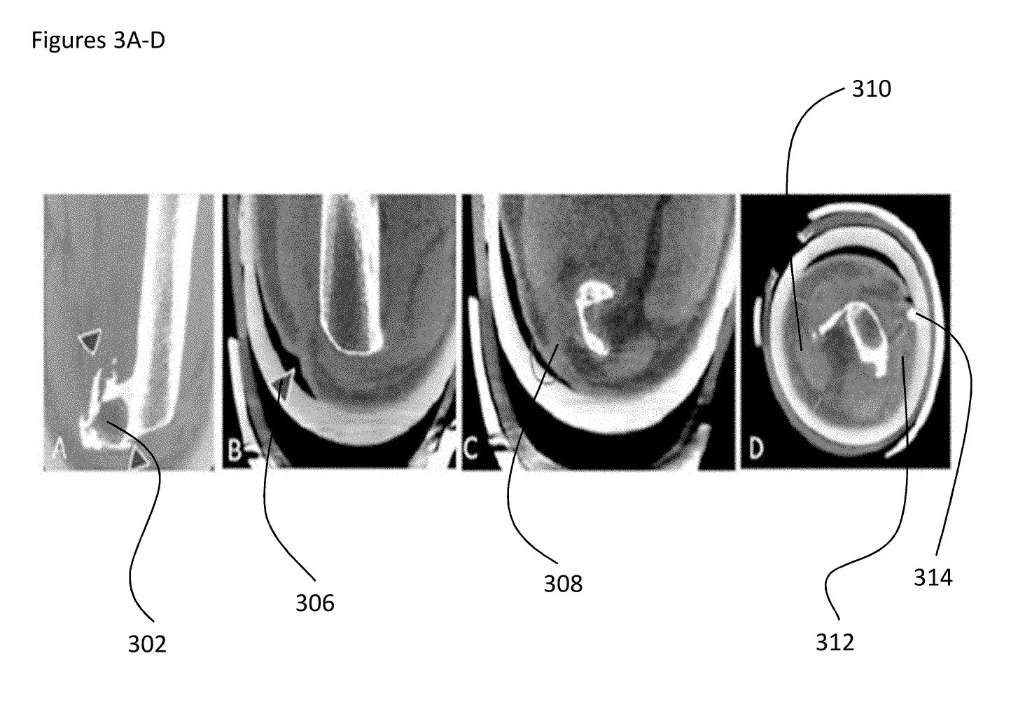

For example, a CT scan of the side of a residual limb is shown in FIG. 2 and illustrates the location of various internal anatomical characteristics, such as bone 204, 206, scar tissue 208, neuroma 210, and bone spur 212. Cross-sectional CT scans commonly provide more detail, such as that shown in FIG. 3A-FIG. 3D. FIGS. 3A-D show common sources of pain in residual limbs. FIG. 3A identifies a location heterotopic ossification 302. FIG. 3B illustrates an example of a retractile skin scar 306, that is, a scar that causes the skin to retract from its original shape). FIG. 3C provides an example of deep tissue scarring 308. FIG. 3D is a CT image that illustrates the distribution and thickness of fat subadjacent to the skin layer. Note that the thickness of fat 310 on one side of the bone is much more substantial than the thickness of fat on the opposite side of the bone 312. Bright spot 314 reflects a metallic ball bearing placed at the site of patient pain.

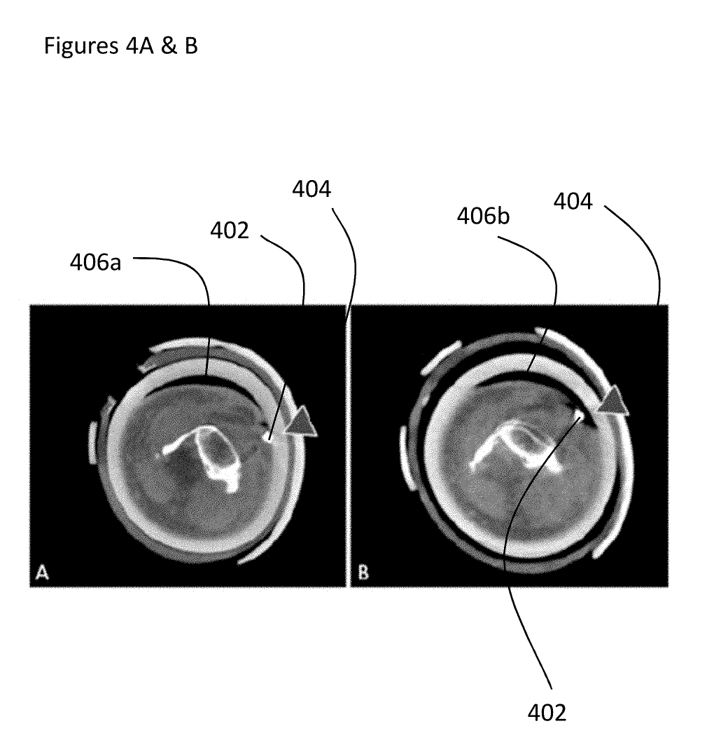

CT scans may also reveal anatomical changes that occur in the remaining limb in weight bearing versus non-weight bearing scenarios, for example as shown in FIGS. 4A and 4B. FIG. 4A is a cross-sectional CT scan of a non-weight bearing residual limb at the level of focal pain 404 experienced by the patient. Again, a metal ball bearing 402 has been placed at the specific site of patient pain. Note that there is an area of gaping 406a between the skin and the liner anteriorly. When the residual limb is weight bearing, as shown in FIG. 4B, that area of gaping 406b increases. This observation suggests that there are repetitive episodes of loading and unloading of weight at this site as the patient ambulates during normal experience.

Thus, the present invention provides a two-part prosthetic socket that may be customized to both internal and external characteristics of the residual limb as well as dynamic information collected while the limb is in use. Within the context of the present invention, the two-part prosthetic socket includes an inner socket component and an outer socket component. The outer socket may be further attached to other prosthetic components. With regard to the descriptions provided herein, “outer” refers to the distal portion of the limb, while “inner” refers to the proximal portion of the limb. Within the context of the present invention, numerous standard and common outer sockets may be employed with the inventive inner socket of the present invention.

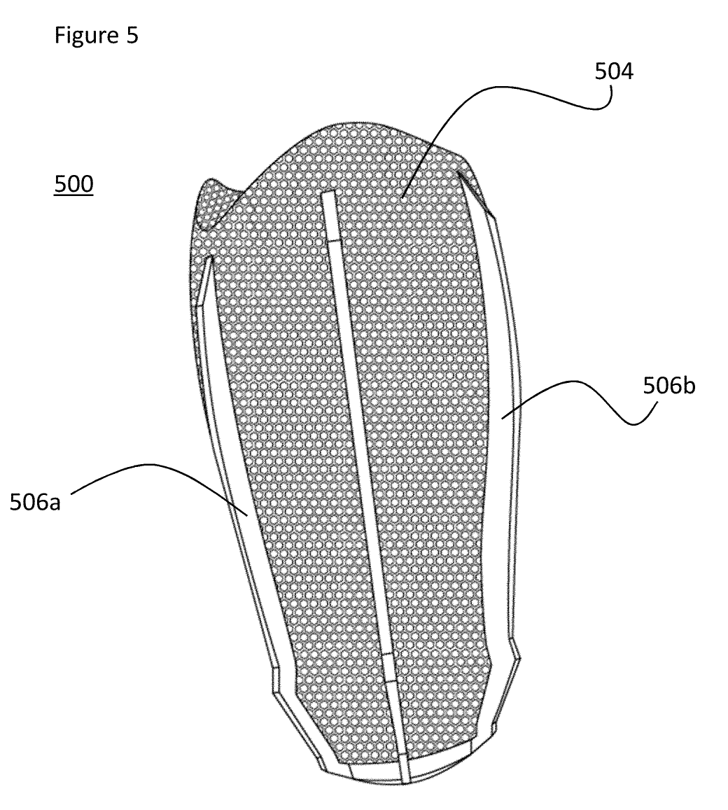

One embodiment of an inner socket component of the present invention is shown in FIG. 5. Within the context of the present invention, the inner socket 500 is designed to be in direct contact with, provide support to, and cushion the residual limb. The inner socket 500 may be fabricated from a material that is both resilient and flexible so that the inner socket 500 conforms to the residual limb. Within the context of the present invention, the material from which the inner socket 500 is fabricated may also be selected so that the inner socket 500 provides cushioning for the residual limb during use by the patient. The inner socket 500 may be prepared by a process that allows flexibility, capacity for cushioning, durability, and thermal management to be tuned based upon the biomechanics of the residual limb as well as feedback from the patient requiring the prosthetic.

Within the context of the present invention, the inner socket 500 may be formed from a flexible material, for example, an elastomeric material. Thus, the inner socket component may accommodate changes in shape, volume, and tone in the residual limb, for example, limb swelling, to provide continual comfort to the patient.

Examples of suitable materials include, but are not limited to, silicon (in particular, those compatible with additive manufacturing processes), thermoplastic elastomers, such as (but not limited to) styrenic block copolymers, polyolefinelastomers, vulcanizates, polyurethanes, copolyester, and polyamides silicon. One of skill in the art would readily be able to recognize a variety of materials that could be utilized to construct an inner socket component by the methods described herein.

In particularly effective embodiments of the present invention, the inner socket 500 is fabricated using a lattice structure. In the embodiment of the inner socket shown in FIG. 5, the lattice employs an open, hexagonal cell structure 504, where the cells are defined by ligaments surrounding the hexagonal voids. In embodiments employing such open-cell structures, the lattice 504 of the inner socket 500 efficiently provides physical support, thermal regulation, and cushioning for the residual limb by being both flexible and breathable. In some embodiments (such as that shown in FIG. 5), the inner socket 500 may include guides 506a, 506b on the outer surface of the inner socket 500. These guides 506a, 506b may align with corresponding guide tracks on an inner surface of an outer socket component (not shown). The corresponding guides and guide tracks improve the likelihood of proper alignment of the inner socket 500 to the outer socket. Additionally, the corresponding guides and guide tracks may also increase the friction between the inner and outer socket components, further improving the stability of the assembly. Further connections points between the inner socket and the outer socket are possible and within the scope of the present invention. For example, a connection may be made between an outer bottom side of the inner socket component and an inner base portion of the outer socket component. One of ordinary skill in the art will be able to accommodate many such structural relationships between an inner socket of the present invention and standard outer sockets that connect to traditional prosthetic limb components.

The lattice structure of the inner socket (shown as hexagonal in FIG. 5) may be varied to achieve desired design objectives, such as greater or lesser flexibility, thermal conduction, or cushioning. Such various design objectives may be achieved in numerous ways, such as by varying the size of the cells that comprise the lattice (e.g., larger cells would generally include larger gaps, leading to generally more pliant properties and greater thermal conduction). Indeed, the present invention encompasses inner sockets having varied lattice structure across the surface of the inner socket, depending on information gathered from various imaging modalities as discussed further below. Furthermore, the properties of the lattice may be varied (either locally or across the entire inner socket) by changing the cellular structure of lattice. For example, different parts of the inner socket component may have different strengths, rigidity, breathability, and durometer by employing differently structured or spaced lattice patterns. The pattern of the lattice structure, for example, body-centered cubic or face-centered cubic, and the shape of each cell (e.g., square, circle, triangular, hexagonal, etc.) will also influence the properties of the material.

Properties of the inner socket may also be tuned by altering the material used to create the lattice pattern. For example, when using polyurethane, the durometer of the polyurethane may be varied by differing the ratio of the base polymers (e.g., isocyanates, polyols), the identity of the base polymers (more/less branching), and the degree of cross-linking. Further, the additional of plasticizers or other additives to affect the density and/or elasticity of the physical properties of the lattice structure. One of ordinary skill in the art will be familiar with well-known methods and techniques to modify the properties of the lattice structure.

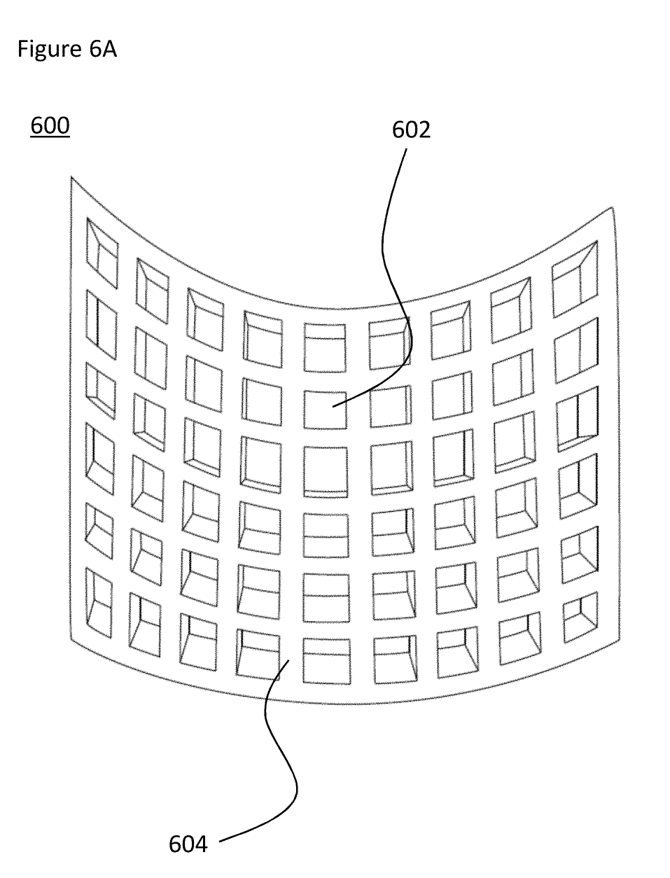

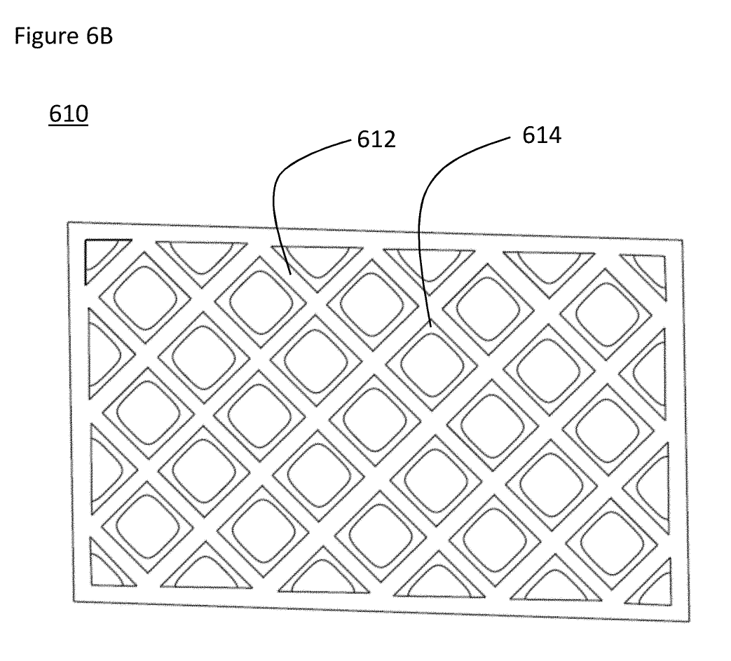

An example of another strategy to achieve flexible and rigid lattice patterns is provided in FIGS. 6A and 6B. The cubic lattice pattern 600 shown in FIG. 6A contains open cells 602 and simple filaments 604. As such, the lattice pattern is flexible and pliant, as shown by its curved structure. The cubic lattice pattern 610 shown in FIG. 6B includes not only the standard filaments 612, but also secondary supporting structures 614 which would improve the rigidity of this portion of the lattice structure of the inner socket.

The inner socket component is preferably in direct contact with and surrounds the residual limb. In some embodiments, a portion of the internal surface of the inner socket component may have a tacky or sticky property that provides a degree of traction between the skin of the residual limb and the inner socket component, aiding to prevent substantial slippage between the inner socket component and the skin of the residual limb. Examples of suitable materials that may be used to achieve an internal tacky surface may be informally referred to as “gels,” and such gels may include any appropriate polymer-based material, such as, but not limited to, urethane, silicon, or thermoplastic elastomer.

In other embodiments, the inner socket component may attach to the residual limb by either conventional pin or suction techniques based on the comfort requirements of the patient.

In another embodiment, the internal surface of the inner socket may include a fluid transport substrate or channeled features that facilitate the flow of moisture or heat. When combined with the breathability of the lattice structure, this may provide an inner socket component with enhanced thermal management for the patient.

Another aspect of the present invention provides a process for manufacturing the inner socket. Briefly, the method includes the steps of
a) receiving digital data defining a three-dimensional (3D) profile of the residual limb, wherein the profile includes internal and external characteristics of the residual limb gathered from at least one imaging technique;
b) processing data collected from those sources to develop a model of an inner socket component; and
c) manufacturing an inner socket component based on the profile derived from the digital data.

Within the context of the present invention, an outer socket component that is releasably attachable about an outer surface of the inner socket component may also be manufactured. Alternatively, commonly available outer sockets may also be coupled with the inner sockets of the present invention.

The digital data received may be from advanced imaging techniques, such as X-ray, computed tomography (CT), magnetic resonance imaging (MRI), or other advanced imaging techniques that assess the inner characteristics of the residual limb. External characteristics include a three-dimensional profile of the residual limb, while internal characteristics include, but are not limited to, scar tissue thickness and location, neuroma size and location, bone spur size and location, heterotopic bone formation, muscle location, bone location, and disproportionate fat distribution, among other physiologically relevant properties. Notably, these internal characteristics contribute substantially to the overall comfort of a prosthesis but are often not measured when using external measuring methods alone.

Medical imaging scans may be performed on the residual limb while the residual limb is resting and while bearing weight. The data collected using this protocol will include the dynamic changes that occur while the prosthetic is in use to inform construction of an inner socket that provides the maximum support and comfort for the residual limbs both at rest and while bearing the patient's weight. For example, performing such measurements will reveal muscle characteristics of the residual limb as well as requirements for operating shape range to ensure retained comfort and fit during ambulation as well as a range of other daily motion.

For example, CT scans of the residual limb may provide the three-dimensional digital profile of the residual limb that includes information including bone size and configuration, muscle size and configuration, scar tissue thickness and location, neuroma size and location, and bone spur size and location, among other physiologically and anatomically relevant features. The digital data may further be utilized to design an operating range for the size and shape of the three-dimensional digital profile of the residual limb. The designed operating range may be based on comparisons of weight bearing and non-weight bearing computed tomography scans.

These advanced imaging techniques provide a three-dimensional (3D) image, such as the one shown in FIGS. 2-4, of the bones, blood vessels, and soft tissues of the body. The 3D image may be used to provide a digital profile of the residual limb that accounts for these features, and which may be adjusted to include an operating range that addresses residual limb volume changes during ambulation, motion, throughout the day, and even over a more-extended span of time. For example, the digital profile may include information from scans of the residual limb in various states, such weight bearing, non-weight bearing, during different times of the day, etc.

The data collected from each type of imaging technique will improve the understanding of the biomechanics of the residual limb and inform the design of the inner socket to achieve maximum support and comfort. As an example, in locations on the residual limb where there is bone or muscle close to the skin surface, greater support may be provided by the inner socket component, e.g., the lattice structure may contain smaller cells and thicker ligaments to provide such greater support or the material from which the lattice is fabricated in those locations may be inherently more rigid. In areas where there is fatty tissue close to the skin surface, the opposite—an inner socket having less support—would be useful to provide the patient with greater comfort. Accommodations in the structure of the inner socket may also be taken when the patient complains of localized pain or neuromas. In this way, the inner socket would not be a uniform lattice shape and structure but would change locally in flexibility, breathability, strength, and durometer to reflect the local biomechanics of the residual limb.

From this data collected by the advanced imaging, a model or blueprint of a custom inner socket can be constructed. In some embodiments of the present invention, a model of a simple socket design can be generated using standard features (e.g., uniform density and durometer throughout inner socket). That initial inner socket design may then be modified using the data collected by advanced imaging, for example to account for surface shape variations and internal anatomical information (e.g., muscle, fat, bone location, density, and structure) may be used to adjust the properties of the designed lattice structures of the inner socket at points where such adjustments would be advantageous to the patient. From the blueprint, the physical inner socket component may be manufactured using standard 3D printing options, as described below.

Initially, inner socket designs may be manually adjusted based upon several 3D views and patient feedback. This may be accomplished using additive manufacturing technologies, for example, by continuous liquid interface processing (CLIP). One skilled in the art of additive manufacturing technologies would be well versed in methods used to manufacture goods utilizing such technology and be able to transform a computer rendition of the inner socket component into the final product without undue experimentation using the mature art of 3D printing. In certain particularly effective embodiments, CLIP technology is utilized, but any technique that is compatible with 3D printing may be utilized within the context of the present invention, such as fused filament fabrication (FFF) with polyurethane filaments. Additionally, the ability to customize the inner socket for each patient (and for the same patient at different points in time) makes 3D printing particularly advantageous for the facile implementation of the present invention.

Feedback, both positive and negative, from patients receiving the latticed inner socket will be utilized to inform future designs. Feedback will include general comfort assessment based, for example, breathability, fit, support as well as specific areas where pain still exist or where local pain caused by an ill-fitting socket was eliminated with a more appropriately customized inner socket to use in the two-part prosthetic socket disclosed herein. Over time, the software will become efficient and effective at designing custom-fit inner sockets for patients with residual limbs displaying varied anatomy and requiring differently fitted and shaped inner sockets.

In further embodiments, the design of the inner socket may be generated initially or modified subsequently utilizing the combination of data derived from radiological scans, artificial intelligence design systems, and patient feedback. There is rapid growth in the field of artificial intelligence and radiology; this growth will enable advances in the design of inner sockets for amputees as well. Initially, inner socket designs may be manually adjusted based upon several 3D views and patient feedback as described above. Those data collected may be provided to an artificial intelligence system that may recognize patterns of improved design characteristics as further data are provided to the artificial intelligence system. As the corpus of patient and radiologic data increase, it is expected that the artificial intelligence system will become more adept at generating improved initial designs. Additionally, in certain embodiments, sensors may be embedded within the inner socket and/or outer socket to collect pressure, motion, vibration, and other local data to further inform the design process.

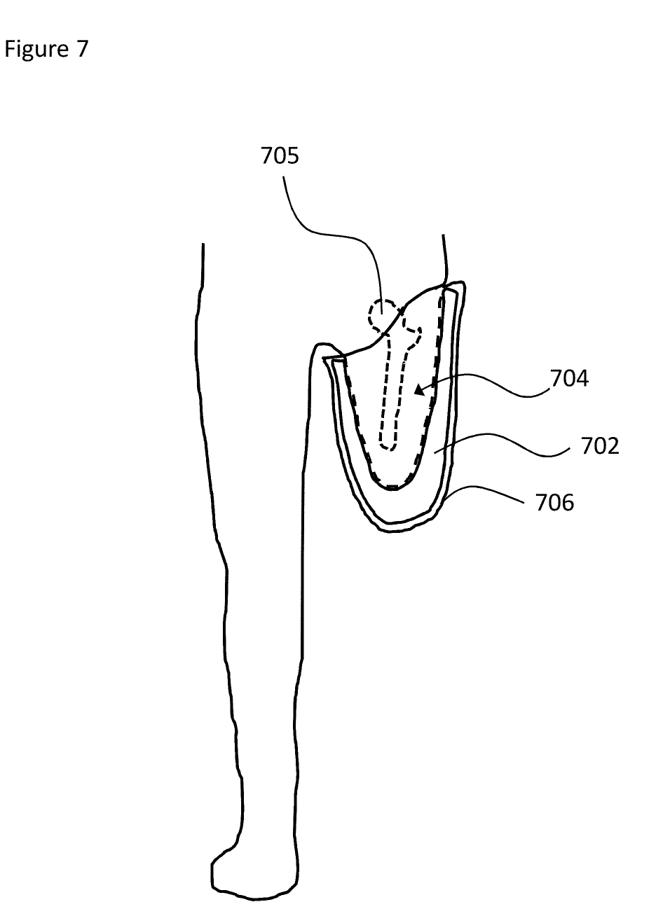

The inner socket component of the present invention preferably interacts with an outer socket component which provides strength and durability where the prosthetic limb is attached to the residual limb via the inner socket. Use of the two-part prosthetic socket in a transfemoral amputation is illustrated as one example in FIG. 7. The residual limb 704 is shown with the remaining portion of the femur 705 also indicated. The inner socket 702 is in direct contact with the residual limb 704 and surrounded by the outer socket 706. The outer socket 706 would then be connected to a traditional prosthetic limb.

Within the context of the invention, the outer socket component may have characteristics similar to and be manufactured by methods well known in the art. For example, the outer socket component may be made from a rigid composite material and thus be both strong and flexible. Current sockets frequently employ carbon fiber, which is very expensive. Due to the characteristics of the inner socket component, the outer socket component may use other composites which offer similar strength with less expense, such as composites which include fiberglass, titanium, aluminum, polycarbonate, high density polyethylene, ultra-high molecular weight polyethylene, poly(methyl methacrylate), etc. In general, the outer socket component may be formed of any composite or material, which may also be ADA compliant and have the strength characteristics deemed appropriate from patient CT data and the overall prosthetic design. The outer socket component may be rigid enough to provide a stable platform, yet may have a degree of adjustability or flexibility (like shoelaces) that allows the two-part prosthetic socket to operate within a range of size and/or shape instead of being designed for a static condition, which is commonly employed in the art. The outer socket component may also incorporate a minor adjustment system, similar to the effect and purpose of the laces of a shoe.

The outer socket component may be manufactured by methods well known in the art, including by continuous liquid interface processing. The two components may be manufactured simultaneously, or may be manufactured separately and assembled prior to use. While the inner socket component is customized to the exact internal and external biomechanics of a patient's residual limb, the outer socket component can be made of a more standard shape, and may be customized or standardized across range of sizes (such as S, M, L, XL). In some embodiments, the friction between the outer surface of the inner socket component and the inner surface of the outer socket component would be sufficient to secure the outer socket component to the inner socket component and prevent slippage or misalignment during usage.

The two-part socket of the present invention includes the outer socket component that is releasably attached to the inner socket component. The manner for releasably attaching the two socket components may be accomplished by a variety of mechanisms. In general, the two-part socket of the present invention is modeled to fit precisely, the outer socket component may be designed to fit around an outer surface of the inner socket component. Relying upon computer-aided design techniques enables an effective interface to be generated between the inner and outer sockets.

The two-part prosthetic socket may attach to a prosthesis by any method well known in the art. For example, there are standard shapes and sizes that are often used by prostheticians. These components are usually pulled from a shelf, and as many as five may be used to connect the socket to the other prosthesis parts, such as artificial knees and feet. Typically, the standard components that provide interconnections in current prosthetics are usually made from titanium to keep them light, but any material may be used. For example, additive manufacturing approaches may be used to manufacture these connections. Lattice structures may also be employed to provide a sturdy, yet light, connection and reduce overall weight of a prosthesis.

The inner socket disclosed and provided herein provide many advantages over the prior art 100 such as the one shown in FIG. 1 that exemplifies a simple outer socket 104 that connects to and is in direct contact with the residual limb and is formed of a hard plastic. This hard plastic socket 104 is often shaped based on a positive mold or from a scan of the outer surface of the patient's residual limb. However, the residual limb often changes over time and changes a socket made of hard unyielding plastic cannot compensate for these changes. Typically, a patient with a new prosthetic will require a new fitting often, possibly as often as every couple of months. This gradually slows to requiring a new fitting every year and even later on, every couple of years.

Conventional techniques for manufacture of prosthetic sockets require weeks (often 6-8 weeks) to perform and are quite labor intensive. However, since the inner socket component disclosed herein may be prepared by processes such as 3D printing, it may be made quickly, for example within hours or days.

The novel designs of the preset invention provide improved comfort by enabling the use of more comfortable designs and materials. This is accomplished by informing the socket fit on measurements of both internal and external properties of the residual limb through imaging techniques and using resilient flexible materials that can be additively manufactured into nearly any imaginable combination to provide a socket with dynamic properties that mirror the needs of the patient's residual limb.

The following non-limiting specific examples presented to illustrate the best mode of carrying out the process of the present invention. The examples are not limited to the particular embodiments illustrated herein but include the permutations, which are obvious set forth in the description.

### EXAMPLES

### Example 1: Use of CT Scanning to Identify Location of Pain in Prosthesis Use

In a first attempt to test whether CT-scan data can aid in the development of a better-fitting socket, we obtained low-dose, cone-beam CT scans from a 52-year old male who experienced a traumatic injury necessitating a transfemoral amputation 2.5 years prior to the scans. The patient experienced pain daily with use of his prosthesis, which started after a few hours of wear and increased throughout the day. The low-dose, cone-beam CT scans were obtained (CurveBeam) with the patient in non-weight bearing and full weight bearing position with the prosthetic inner socket and outer socket in place. Full weight bearing was a static condition meant to simulate pressures during normal gait. A small metallic marker (BB) was physically placed on the site of pain as indicated by the patient to mark the site on the CT scan. Data analysis: DICOM images were post-processed to reduce noise, and loaded into DICOM-viewing software (Osirix) for image viewing. The following characteristics known to be potential causes of pain were identified in relation to the pain marker: heterotopic ossification (exuberant bone growth), scar location, surface irregularities on the residual limb, deep scar tissue involving fascia, and distribution of fat relative to soft tissue. Additionally, the overall area of tissue interfacing with the inner socket and outer socket was assessed in non-weight bearing and weight bearing status.

Results: There was a difference in residual limb to prosthesis contact between non-weight bearing and weight-bearing status, and this correlated with the site of pain. Specifically, there was a gap between the focal pain site and the prosthesis in weight bearing, and this gap was closed at rest. This suggests that there are repeated episodes of pressure loading and unloading in one focal area where the patient experiences pain.

There was additionally scar tissue and deeper scar involving the fascia at the site of pain. While the patient did have heterotopic ossification, this was remote from the site of pain and was therefore considered non-contributory.

While specific embodiments of the invention have been described in detail, it should be appreciated by those skilled in the art that various modifications, alternations, and applications could be developed in light of the overall teachings of the disclosure. Accordingly, the particular arrangements, systems, apparatuses, and methods disclosed are meant to be illustrative only and not limiting as to the scope of the invention.

What is claimed is:

1. A method for providing a prosthetic inner socket, comprising
a) receiving digital data collected from an imaging modality regarding three-dimensional (3D) properties of a residual limb, wherein the digital data includes internal and external characteristics of the residual limb;
b) processing said digital data to develop a digital profile of the inner socket; and
c) manufacturing the inner socket based on the digital data.

2. The method according to claim 1, wherein the imaging modality is selected from the group consisting of X-ray, CT, MRI, and combinations thereof.

3. The method of claim 1, wherein the manufacturing step is accomplished using 3D printing.

4. The method of claim 1, wherein the digital data includes information selected from the group consisting of external dimensions of the residual limb, internal characteristics of the residual limb, and combinations thereof.

5. The method of claim 4, wherein the internal characteristics of the residual limb are selected from the group consisting of scar tissue thickness and location, neuroma size and location, bone spur size and location, muscle location, bone location, fatty tissue location, and combinations thereof.

6. The method of claim 4, wherein the digital data is collected from the residual limb when the residual limb is at rest and when the residual limb is in weight-bearing use.

7. The method of claim 6, further comprising a step of designing the inner socket to accommodate the digital data collected from the residual limb when the residual limb is at rest and when the residual limb in weight-bearing use.

8. The method of claim 7, wherein the designing step includes the selection of a lattice structure of the inner socket that incorporates information from the digital data.

9. The method of claim 8, wherein the inner socket is formed from a flexible material.

10. The method of claim 9, wherein the flexible material is an elastomeric material.

11. The method of claim 10, wherein the elastomeric material forms a lattice further comprised of cells and ligaments, wherein the lattice pattern is selected from the group consisting of cubic, circular, triangular, hexagonal, and combinations thereof.

12. The method of claim 1, wherein the processing of the data is done by artificial intelligence that combines structured and unstructured data from the imaging modality, feedback from the patient who will utilized said prosthetic socket, biometric information selected from the group consisting of blood pressure, body weight, fat composition, and combinations thereof, and data collected from a medical practitioner selected from the group consisting of gait analysis, medical examination, and biodynamic examination

13. An inner socket of a prosthetic device adapted to a residual limb, wherein the inner socket has a shape complementary of the residual limb of a patient, said inner socket comprising a lattice comprised of cells and ligaments and wherein the cells and ligaments are designed based upon digital data collected from an imaging modality regarding three-dimensional (3D) properties of the residual limb.

14. The inner socket component according to claim 13, the imaging modality is selected from the group consisting of X-ray, CT, MRI, and combinations thereof.

15. The inner socket according to claim 13, wherein the digital data includes information selected from the group consisting of external dimensions of the residual limb, internal characteristics of the residual limb, and combinations thereof.

16. The inner socket according to claim 15, wherein the internal characteristics of the residual limb are selected from the group consisting of scar tissue thickness and location, neuroma size and location, bone spur size and location, muscle location, bone location, fatty tissue location, and combinations thereof.

17. The inner socket according to claim 13, wherein the digital data is collected from the residual limb when the residual limb is at rest and when the residual limb is in weight-bearing use.

18. The inner socket according to claim 13, wherein the inner socket comprises an elastomeric material.

19. The inner socket according to claim 13, wherein lattice has a pattern selected from the group consisting of square, circle, triangular, hexagonal, and combinations thereof.

20. The inner socket according to claim 13, wherein the design of the lattice is based on the digital data.
